(19) United States
(12) Patent Application Publication
(10) Pub. No.: US 2007/0117867 A1
(43) Pub. Date: May 24, 2007

(54) Kit comprising three independent compositions and methods for building muscle, increasing strength, increasing muscle size, increasing muscle performance and reducing muscle fatigue

(76) Inventors: Paul T. Gardiner, Mississauga (CA); Marvin A. Heuer, Mississauga (CA)

Correspondence Address: KENYON & KENYON LLP, ONE BROADWAY, NEW YORK, NY, 10004, US

(21) Appl. No.: 11/489,116

(22) Filed: Jul. 18, 2006

Related U.S. Application Data
(60) Provisional application No. 60/735,057, filed on Nov. 8, 2005.

(51) Int. Cl. A61K 31/195; A01N 37/12

(52) U.S. Cl. 514/565

(57) ABSTRACT

A kit, and a method of using same, that comprises first, second and third components wherein the first, second and third components collectively comprise Glycine, Arginine, Leucine, Creatine, Alpha Lipoic Acid, and earth metal salts of Ketoisocaproic Acid. Use of the kit may enhance muscle performance, build muscle, increase muscle strength, increase muscle size and reduce muscle fatigue. The three compositions may be used in synergy so as to promote muscle anabolism, neutralize reactive oxygen species, and improve creatine uptake in muscle tissue. The method may also include a schedule for administering each composition to the user for optimal results.

### RELATED APPLICATIONS

This application is related to and claims benefit of priority to Applicant's co-pending U.S. Provisional Patent Application Ser. No. 60/735,057 entitled “Supplemental Dietary Composition for Turning on Anabolic Switches in Muscle, Stimulating and/or Optimizing Protein Synthesis, and/or Potently Signaling Muscle Building and/or Growth,” filed Nov. 8, 2005, the disclosure of which is fully incorporated by reference.

### FIELD OF THE INVENTION

The present invention relates to a kit comprising three independent compositions, wherein each one of the compositions is administered at least once daily for the overall purposes of enhancing muscle performance, building muscle, increasing muscle strength, increase muscle size and reduce muscle fatigue.

### BACKGROUND OF THE PRESENT INVENTION

It may be desirable to create an environment which permits high levels of resistance training for the purposes of building muscle, increasing strength, increasing muscle size, increasing muscle performance and reduce muscle fatigue in the area of athletic performance. However, high levels of training intensity, e.g., acute, intense anaerobic use of skeletal muscle, often results in impaired athletic performance. Associated with impaired athletic performance is the generation of lactate, ammonium and hydrogen ions. These by-products of acute, intense resistance exercise are characteristic of muscle fatigue. The reversal of these metabolic fatiguing factors is important for recovery from exercise as they can lead to a slowing or inhibition of a performance athlete's ability to train to the required levels for their respective disciplines.

It is known that exhaustive exercise can diminish energy sources in the muscle and lead to the disruption of the skeletal muscle nitrogen balance, which may result from the body's need to provide energy for continued exercise. In order that they may be used a fuel sources, certain amino acids, such as branched-chain amino acid, are released from muscle and are deaminated, resulting in a molecule which can be used as a source of energy. However, this also elevates serum nitrogen levels and contributes to acidosis. This process leads to muscle fatigue and impaired recovery from exercise. Furthermore, the release of amino acids from skeletal muscle as an energy source also contributes to muscle catabolism, thereby resulting in an environment that is unfavorable to building muscle and increasing strength. Concomitant with this, there is a decrease in protein synthesis, again hindering muscle building and increasing strength. Additionally, with increasing levels of serum nitrogen, a decrease in catalytic efficiency of contractile events is observed.

The removal of nitrogen by-products, the restoration of nitrogen balance by the reamination of Ketoisocaproic Acid to Leucine, and the obstruction of the catabolic protein energy deriving pathways may provide for building muscle, increasing strength, increasing muscle size, increasing muscle performance and reducing muscle fatigue.

Supplementation of certain branched-chain amino acids has also been shown to activate anabolic pathways in the muscle. The activation of these pathways leads to the activation of a cellular initiation complex, resulting in muscle growth via increased protein synthesis. It has recently been demonstrated that key amino acids, notably branched-chain amino acids such as Leucine, are able to regulate mRNA translation and can be used to directly activate protein synthesis for the purposes of muscle growth and development (Yoshizawa F. Regulation of protein synthesis by branched-chain amino acids in vivo. Biochem Biophys Res Commun. 2004 Jan 9;313(2):417-22. Review). Moreover, by supplying amino acids, catabolism of skeletal muscle to be used as an energy source is greatly reduced, thereby shifting the muscle building process in favor of anabolism.

Increasing the ability of the body to produce Adenosine Triphosphate (ATP) may also play a role in building muscle and increasing muscle strength. ATP is the energy currency of the cell; and it is used to produce muscle contractions (Campbell N A, Biology 3rd ED. The Benjamin/Cummings Publishing Company, Inc. Don Mills, Ontario. 1993). Following ATP's use in muscle contractions, it is reduced to Adenosine Diphosphate, which is devoid of energetic properties with respect to muscle contraction. Creatine is able to bind the removed phosphate from ATP following muscle contractions. It is then able to rapidly donate the phosphate to the resultant ADP (Gaitanos G C, Williams C, Boobis L H, Brooks S. Human muscle metabolism during intermittent maximal exercise. J Appl Physiol. 1993 Aug;75(2):712-9.), thereby generating ATP to be used in subsequent contractions.

### SUMMARY OF THE INVENTION

The present invention provides a kit comprised of three independent compositions and also provides a method of employing the kit, e.g., the compositions. The present invention may provide a method for one or more of building muscle, increasing strength, increasing muscle size and increasing muscle performance by one or more of decreasing muscle protein catabolism, increasing anabolism, maintaining favorable conditions of serum nitrogen, and augmenting the muscular energy source before, during and post-exercise.

The present invention, according to various embodiments, thereof pertains to a kit comprising at least Glycine, Arginine, Leucine, Creatine, Alpha Lipoic Acid, and earth metal salts of Ketoisocaproic Acid. The kit may be comprised of three independent compositions, wherein each composition may be administered to an individual, e.g. a human or an animal, for the purposes of one or more of enhancing muscle performance, building muscle, increasing muscle strength, increasing muscle size and reducing muscle fatigue. In addition, the present application also relates to a method which may include the step of, e.g., administering said kit to an individual, e.g. a human or an animal, for the purposes of one or more of enhancing muscle performance, building muscle, increasing muscle strength, increasing muscle size and reducing muscle fatigue.

### DETAILED DESCRIPTION OF THE INVENTION

The present invention provides a kit, the use of which may provide a method for enhancing muscle performance under conditions of acute anaerobic resistance training. The use of the kit may further provide a method which provides for the synergistic functioning of three independent compositions for the purposes of one or more of enhancing muscle performance, building muscle, increasing muscle strength, increasing muscle size and reducing muscle fatigue.

A first composition of the kit may comprise, for example, Glycine, L-Arginine and Alpha-ketoisocaproic acid. The first composition may be used to enhance muscle performance, and recovery from fatigue in an individual, e.g. a human or an animal. For example, U.S. Pat. No. 6,100,287, the disclosure of which is fully incorporated by reference herein, describes a method for enhancing muscle performance and recovery from fatigue wherein the method includes administering a composition of a cationic or dibasic amino acid and a ketoacid. A specific embodiment disclosed therein comprises Glycine, and the L-Arginine monohydrochloride salt of Alphaketoisocaproic acid calcium.

The aforementioned formulation, e.g., Glycine, and the L-Arginine monohydrochloride salt of Alphaketoisocaproic acid calcium, has benefits which may include the following:

1) A more rapid and complete recovery from intense acute athletic training and greater muscle power;

2) It provides a safe way to enhance athletic performance since the composition of Glycine, and the L-Arginine monohydrochloride salt of Alphaketoisocaproic acid calcium is devoid of the detrimental side effects of anabolic steroids; and

3) The composition of Glycine, and the L-Arginine monohydrochloride salt of Alphaketoisocaproic acid calcium can improve exercise tolerance and recovery during rehabilitation.

A second composition of the kit may comprise, for example, Leucine or monobasic branched—chain amino acids and Ketoisocaproic Acid. The second composition of the kit may be used to stimulate and optimize protein synthesis, potently signal muscle building and increase muscle size in an individual, e.g., a human or an animal. For example, Applicant's co-pending Provisional Patent Application No. 60/1735,057, discloses compositions and methods to directly activate protein synthesis for the purposes of muscle growth and development which may be employed in the second composition of the kit. Leucine and other branched-chain amino acids are able to directly modify critical points (Fingar D C, Richardson C J, Tee A R, Cheatham L, Tsou C, Blenis J. mTOR controls cell cycle progression through its cell growth effectors S6K1 and 4E-BP1/eukaryotic translation initiation factor 4E. Mol Cell Biol. 2004 Jan;24(1):200-16.) in muscle development to activate the protein kinase mTOR (mammalian target of rapamycin); a site of integration of the signals that stimulate muscle protein synthesis, cell growth and size as well as progression into the cell cycle.

The second composition of the kit, e.g., Leucine and Ketoisocaproic Acid, may be designed to influence the genetic control system for muscle protein synthesis to stimulate muscle growth, increase muscle mass, decrease catabolic, decrease recovery time and treat muscle wasting.

A third composition of the kit may comprise, for example, Creatine, Alpha Lipoic Acid and Ketoisocaproic Acid. The third composition may be used to neutralize reactive oxygen species and improve creatine uptake into muscle tissue.

Alpha Lipoic Acid is an insulin modulator and an antioxidant that serves as protection against oxidative injury in non-neuronal and neuronal tissue (Bloch-Damti A, Bashan N. Proposed mechanisms for the induction of insulin resistance by oxidative stress. Antioxid Redox Signal. 2005 Nov-Dec;7(11-12):1553-67. Review.) Alpha Lipoic Acid is a nutrient that the human body makes in minute quantities and may be obtained from yeast and liver (Wollin S D, Jones P J. Alpha-lipoic acid and cardiovascular disease. J Nutr. 2003 Nov;133(11):3327-30. Review.) Studies have shown that Alpha Lipoic Acid can significantly increase the body's utilization of blood sugar in type II diabetics and that Lipoic Acid may increase the metabolic clearance rate of glucose. Moreover, Alpha Lipoic Acid may improve creatine uptake in the body by a similar mechanism.

Creatine is a naturally occurring amino acid derived from the amino acids glycine, arginine, and methionine. It is readily found in meat and fish and is also synthesized by humans. The main role of creatine is as a fuel renewal source in muscle. About 65% of creatine is stored in muscle as Phosphocreatine (creatine bound to a phosphate molecule) (Casey A, Constantin-Teodosiu D, Howell S, Hultman E, Greenhaff P L. Metabolic response of type I and II muscle fibers during repeated bouts of maximal exercise in humans. Am J Physiol. 1996 Jul;271(1 Pt 1):E38-43). Phosphocreatine serves as a major source of phosphate wherein ADP is able to bind said phosphate to re-generate to form ATP which can be used in subsequent contractions. After 6 seconds of exercise, muscle concentrations of Phosphocreatine drop by almost 50% (Gaitanos G C, Williams C, Boobis L H, Brooks S. Human muscle metabolism during intermittent maximal exercise. J Appl Physiol. 1993 Aug;75(2):712-9.) as it is used to regenerate ATP. Creatine supplementation has been shown to increase the concentration of Creatine in the muscle (Harris R C, Soderlund K, Hultman E. Elevation of creatine in resting and exercised muscle of normal subjects by creatine supplementation. Clin Sci (Lond). 1992 Sep;83(3):367-74.) and increase the resynthesis of Phosphocreatine within 2 minutes of recovery following exercise (Greenhaff P L, Bodin K, Soderlund K, Hultman E. Effect of oral creatine supplementation on skeletal muscle phosphocreatine resynthesis. Am J Physiol. 1994 May;266 (5 Pt 1):E725-30.).

Advantageously, each of the three aforementioned compositions of the kit may perform different functions. By providing the three compositions in the form of a kit, a daily regimen of the compositions can advantageously and conveniently be administered in an effective sequence so as to enhance muscle performance, build muscle, increase muscle strength and increase muscle size at a time wherein each of the said compositions may be optimally effective.

The method of using the kit involves the administration to an individual, e.g., a human or an animal, of each one of the compositions of the kit at different times of the day as well as in relation to an individual's workout schedule. The first independent composition of the kit, comprised of Glycine, and the L-Arginine monohydrochloride salt of Alphaketoisocaproic acid calcium, may be administered to an individual, by having the individual ingest the composition. On days when the individual participates in exercise, the first composition may be administered up to 45 minutes, e.g., 30 minutes, prior to exercise.

Furthermore, said second independent component of the kit, comprised of, e.g., Leucine and Ketoisocaproic Acid, may be administered to an individual, by having the individual ingest the composition on a daily basis. On days when exercise will take place, the daily serving of said second component of the kit is administered prior to exercise. On days where exercise will not take place, the serving is administered in the morning.

Additionally, said third independent component of the kit, comprised of, e.g., Creatine, Alpha Lipoic Acid and Ketoisocaproic Acid may be administered to an individual, by having the individual ingest the composition on a twice daily basis. The servings of said third independent component of the kit may be administered twice daily, wherein the first serving is administered in the morning on both exercise and non-exercise days, followed by a second serving which may be administered post-exercise. On days where exercise will not take place, the second serving is to be administered in the afternoon.

Although the following example illustrates the practice of the present invention in one of its embodiments, the example should not be construed as limiting the scope of the invention. Other embodiments will be apparent to one skilled in the art from consideration of the specification of the following example.

### EXAMPLE

As an example, a 15-day regimen is used to illustrate the dosing protocol of the 3 independent compositions of said kit of the present invention. It should be noted that a regimen of any length may be used. An exercise training program may be followed on certain days of the regimen. On these days, the composition of the kit may be administered according to following regimen: 1 serving of a composition comprising Creatine, Alpha Lipoic Acid and Ketoisocaproic Acid in morning, 1 serving of a composition comprising Glycine, and the L-Arginine monohydrochloride salt of Alphaketoisocaproic acid calcium up to 45 minutes, e.g., 30 minutes, prior to exercise, 1 serving of a composition comprising Leucine and Ketoisocaproic Acid post-exercise and 1 serving of a composition comprising Creatine, Alpha Lipoic Acid and Ketoisocaproic Acid post-exercise. On other days the individual may refrain from undertaking the exercise training program. On these days, wherein an exercise training program is not undertaken, the components of the kit may be administered according to the following regimen: 1 serving of a composition comprising Creatine, Alpha Lipoic Acid and Ketoisocaproic Acid in the morning, 1 serving of a composition comprising Leucine and Ketoisocaproic Acid in the morning, 1 serving of a composition comprising Creatine, Alpha Lipoic Acid and Ketoisocaproic Acid in the afternoon.

What is claimed:

1. A method comprising the steps of:
administering to a user a kit, the kit comprising first, second and third components wherein the first, second and third components collectively comprise Glycine, Arginine, Leucine, Creatine, Alpha Lipoic Acid, and earth metal salts of Ketoisocaproic Acid.

2. The method of claim 1, wherein said first component comprises Glycine, Arginine and Ketoisocaproic Acid, the first component being administered to the user up to 45 minutes prior to a workout.

3. The method of claim 1, wherein said second component, comprises Leucine and Ketoisocaproic Acid, the second component being administered to the user at least once daily.

4. The method of claim 1, wherein said second component comprises Leucine and Ketoisocaproic Acid the second component being administered to the user immediately prior to a workout.

5. The method of claim 1, wherein said third component comprises Creatine, Alpha Lipoic Acid and Ketoisocaproic Acid, the third component being administered to the user at least once daily.

6. The method of claim 1, wherein the third component is administered upon waking in the morning.

7. The method of claim 1, wherein said third component comprises Creatine, Alpha Lipoic Acid and Ketoisocaproic Acid, the third component being administered to the user immediately following a workout.

8. The method of claim 1, wherein the first, second and third components collectively comprise Glycine, Arginine, Leucine, Creatine, Alpha Lipoic Acid, and earth metal salts of Ketoisocaproic Acid in amounts effective to one or more of enhance muscle performance, build muscle, increase muscle strength, increase muscle size and reduce muscle fatigue.

9. A kit comprising:
first, second and third components, each one of the first, second and third components being configured for ingestion by a user at a predetermined time of day, wherein the first, second and third components collectively comprise Glycine, Arginine, Leucine, Creatine, Alpha Lipoic Acid, and earth metal salts of Ketoisocaproic Acid.

10. The kit of claim 9, wherein said first component comprises Glycine, Arginine and Ketoisocaproic Acid, the first component being configured for ingestion by the user up to 45 minutes prior to a workout.

11. The kit of claim 9, wherein said second component comprises Leucine and Ketoisocaproic Acid, the second component being configured for ingestion by the user at least once daily.

12. The kit of claim 9, wherein said second component comprises Leucine and Ketoisocaproic Acid the second component being configured for ingestion by the user immediately prior to a workout.

13. The kit of claim 9, wherein said third component comprises Creatine, Alpha Lipoic Acid and Ketoisocaproic Acid, the third component being configured for ingestion by the user at least once daily.

14. The kit of claim 13, wherein the third component is configured for ingestion by the user upon waking in the morning.

15. The kit of claim 13, wherein the third component is configured for ingestion by the user immediately following a workout.

16. The kit of claim 9, wherein the first, second and third components collectively comprise Glycine, Arginine, Leucine, Creatine, Alpha Lipoic Acid, and earth metal salts of Ketoisocaproic Acid in amounts effective to one or more of enhance muscle performance, build muscle, increase muscle strength, increase muscle size and reduce muscle fatigue.

17. A method for at least one of enhancing muscle performance, building muscle, increasing muscle strength, increase muscle size and reduce muscle fatigue, the method comprising the steps of administering to a user at least once daily:
a first composition of Glycine, Arginine and earth metal salts of Ketoisocaproic Acid;
a second composition of Leucine and earth metal salts of Ketoisocaproic Acid; and
a third composition of Creatine, Alpha Lipoic Acid and earth metal salts of Ketoisocaproic Acid.

18. The method of claim 17, wherein the first, second and third compositions are administered in the form of a kit.

19. The method of claim 18, wherein the step of administering the kit is performed as part of a 15-day regimen.

20. The method of claim 19, wherein the 15-day regimen further comprises the step of the user performing physical exercise.

21. The method of claim 19, wherein amounts of said first, second and third compositions are sufficient to cause, when combined with the performance of the physical exercise, a change in body composition in the user.
